(19) United States
(12) Patent Application Publication
(10) Pub. No.: US 2006/0113486 A1
(43) Pub. Date: Jun. 1, 2006

(54) Reaction chamber

(75) Inventors: Edgar B. Dally, Monterey County, CA (US); James C. Paul, Monterey County, CA (US)

Correspondence Address: Stanley Z. Cole, 26620 St. Francis Road, Los Altos Hills, CA, 94022, US

(73) Assignee: VALENCE CORPORATION

(21) Appl. No.: 10/998,105

(22) Filed: Nov. 26, 2004

(51) Int. Cl. A61N 5/00

(52) U.S. Cl. 250/492.3

(57) ABSTRACT

A novel reaction chamber is described for the treatment of gases which either have a noxious odor or include toxic elements. The chamber is for treatment of the gases with energetic electrons and uses an extended electron source in the center of a chamber volume which creates electrons that move out of the source and radially into the chamber. The gases are flowed into the area of the source and away from the source as to result in uniform and efficient exposure of the flowing gases.

### FIELD OF THE INVENTION

This invention has to do with reaction chambers or reaction volumes for efficient energy transfer to gases. In particular, the objective is to optimize the exposure of contaminated gases to energetic electrons generated by a symmetrical electron beam source shaped as an extended cylinder.

### BACKGROUND OF THE INVENTION

Energetic electrons are used today to neutralize toxic gases and to reduce noxious odors. Although some interest has been expressed in the shape of the chamber in which such treatments occur, in fact to date, the emphasis has not been one of great interest in optimizing chambers for efficient treatment. Various patents exist in this field and some do discuss the reaction chambers. For example in U.S. Pat. No. 5,319,211, a detoxification plenum or tank is illustrated in which electrons attack gases moving through. Also referred to in that patent are U.S. Pat. Nos. 4,507,265; 5,015,443; 4,569,642; and 4,915,916, patents all dealing with the reduction of toxic flue gases generally found in power plants where massive structures are used for treatment of effluent stack gases. These gases are generally SOₓ and NOₓ or organics with toxics present. A similar plenum to the one illustrated in U.S. Pat. No. 5,319,211 is also shown in U.S. Pat. No. 5,357,291. The chamber in this latter patent is illustrated as transportable and is discussed with supplemental treatment stations to clean fluids primarily gaseous in nature. All of these are generally large and cumbersome and none were created for efficient transfer of energetic electrons. Accordingly, neither the environmental nature of the gas nor the smell of the gas is improved as much as might be done if an improved transfer of energetic electrons could be achieved. Since the technique of treating gases with energetic electrons is in early stages of development, only limited interest has been shown in shaping chambers to achieve efficient radiation of gas flows subjected to treatment or to create chambers of small sizes as to enable systems to fit into more enclosed areas as to simplify the fit of systems within enclosures.

A different structure, which can be considerably smaller than those illustrated in the patents that have been discussed, is illustrated and described in U.S. Pat. No. 5,378,898. In this case, the chamber was designed to receive electrons spewed out of the end of an electron source which although operative for the system shown may not be the best combination of generator and chamber for efficient treatment of toxic or noxious smelling gases.

### SUMMARY OF THE INVENTION

This invention is concerned with the optimization of equipment and processes to expose contaminated gas flows to energetic electrons generated by an electron beam source shaped like an extended cylinder. The source which has been developed for this application may comprise a vacuum tube of the type described in pending U.S. patent application Ser. No. 10/822,890, the disclosure of which is incorporated herein by reference. This device has the unique quality of emitting electrons circumferentially. The emitted electrons are caused to interact and/or bombard carrier gases, typically air, that contain or carry undesirable contaminating compounds. The objective is to convert the contaminant compounds into compounds that are more environmentally acceptable from the point of view of regulations, health and/or smell. The carrier gas with contaminants is flowed through a conduit and directed into a chamber which may be called a reaction chamber or a reaction chamber volume. Electrons produced within a vacuum are accelerated by an applied voltage and are caused to pass through a thin window of the vacuum unit into the reaction volume where they interact through electromagnetic processes with the compounds in the flowing gas. This form of interaction produces a chain of chemical reactions meant to reduce undesirable compounds to more acceptable compounds or bad smells to acceptable ones.

In order to effectively treat a fluid flow, it is important to design the reaction chamber such that the available electron energy is transferred as efficiently as possible to the incoming atoms in order to initiate chemical reactions leading to the optimum level of conversion in time and quantity of the contaminating compounds.

Electrons have a relatively short range in gases, depositing a large amount of their energy more or less uniformly within a short distance, then losing the balance of energy in a short distance thereafter. Because of this, it is important that the gas to be treated flows through that volume of a reaction vessel where most of the energy of the electrons is deposited. Thus an objective is to deposit the energy per unit volume as uniformly as possible into each fraction of the moving gas. To achieve this objective, the gas must be forced to flow through the region of high electron intensity. As this occurs electrons collide with molecules and alter the compounds in the gas to form radicals that initiate reactions that will reduce the contaminants in the gases themselves. This is achieved in large measure through the design of the reaction chamber of this invention.

Other factors that enter into the reaction volume design are the need for radiation shielding from X-rays produced both by the electron source itself and in the reaction volume structure, and to minimize the pressure drop and dynamic pressure of the gas flowing through the chamber. Dimensions of air flow paths within the reaction volume need to be designed to accommodate the flow to minimize flow resistance and dynamic pressure of the flowing gas. These considerations also impact the size of blowers or compressors used to force gas through the reaction volume at the required rate. One of the objectives of this design is to reduce flow resistance through the reaction volume to less than that presented by connecting input and output piping. Thus typical gas fluid dynamic calculations may be applied to determine the dimensions required to meet these criteria.

### BRIEF DESCRIPTION OF THE DRAWINGS

FIG. 1 is a schematic drawing of an embodiment of a top view of a reaction chamber volume in which upper cover is removed.

FIG. 2 is cutaway schematic view of an internal section of the reaction chamber.

FIG. 3 is a cutaway schematic of another internal section of the reaction chamber.

### DETAILED DESCRIPTION OF THE INVENTION

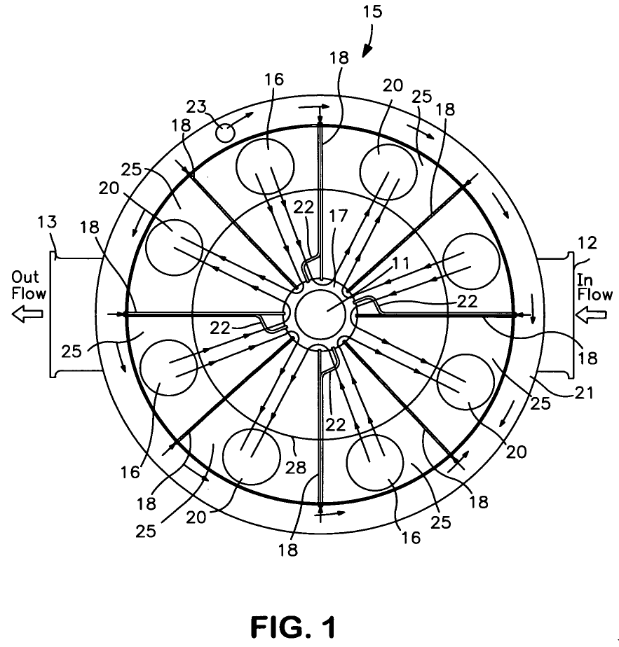

The geometry of the electron source is a major factor in how the chamber should be designed in order to accomplish the objectives of efficient treatment of a flowing gas. In this instance, a cylindrical source is used since it appears to offer the greatest possibility of a highly efficient treatment system as will be further described herein. The reaction chamber is optimized for an extended cylindrical source as shown in FIG. 1 or for a cylindrical source like that discussed in FIG. 1. The source may be segmented around its periphery, either into a single segment of any angular extension, on into symmetrically or asymmetrically arranged segments of any chosen angular segment or segments. Such a source is fully described in pending application Ser. No. 10/822,890 incorporated herein by reference above. The preferred source for this invention is one that provides an output around its entire periphery.

For a cylindrical source of electrons, the preferred approach is to cause maximum energy transfer or power transfer to the incoming gas by directing the incoming gas directly, radially, toward the source around the 360 degree circumference of the source. The gas, after flowing to the source, is then diverted to an adjacent radially located chamber to carry the flow out of the reaction volume. This approach is shown and described in connection with the Figures that follow.

The approach chosen in the embodiment illustrated is to direct the flow radially inward, turn it around and flow it radially outward on a separate path. This is accomplished by dividing the cylindrical volume around the cylindrical source into pie-shaped segments, flowing gas inward in one pie shaped section, turning the flow in that section at the source and flowing the gas to the next section and then outwardly away from the center. Controlling the process gas flow in this manner exposes the flowing gas being fed to the chamber for treatment to the entire space distribution of the beam.

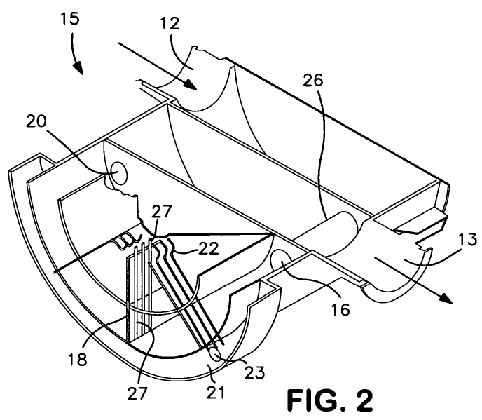

Referring now to FIG. 1, a reaction chamber 15 is illustrated (with the cover removed) and includes a central source for energetic electrons which may comprise an electron beam source 11 such as a vacuum tube to generate electron beams disclosed and described in pending patent application Ser. No. 10/822,890 or may comprise other sources capable of emitting energetic electrons in a radial pattern from an extended cylindrical central element or central area or volume in chamber 15. This for example in a small unit could comprise a corona generator such as an elongated wire to which a high voltage is applied. The materials to be treated which generally will be a fluidic flow of gaseous compounds are fed into the reaction volume through conduit 12 and after treatment within the reaction volume exit from chamber 15 through conduit 13. Connected to the reaction volume are input openings 16 to convey the incoming gases for treatment into a pie shaped segment of the reaction volume in an effective zone of the charged particles. These openings 16 are connected to the input conduit 12 through plenum 26 (shown in FIG. 2) so that an inflow of gas to be treated travels into the input conduit 12, through plenums 26 and out of openings 16 into the reaction volume for treatment purposes. Gases from openings 16 are directed to the center area where electron source 11 is located. The input flow as it reaches or just about reaches the source is directed into path 17 adjacent to and around source 11. The gases move as directed by vanes 18 to and against source 11 and then back through an adjacent pie shaped section 25 and out through output openings 20. Thus gas flow from the inlet openings 16 travels to source 11 and then to outlet openings 20 on either side of the subject inlet pie section 25. Openings 20, through which the outflowing gases travel from the reaction volume, in turn, are connected to output conduit 13 from which the gases leave reaction chamber 15.

Contaminated air flowing in and out through plenum connecting tubes to openings 16 and/or 20, pass through a perforated cylinder 28 on the path toward and away from the electron source. This perforated wall is for the purpose of smoothing the air flow and uniformly distributing air during its path to and from the electron source.

Along the outer wall of the circumference of the illustrated chamber 15, is coaxially positioned passageway 21, a separate, narrow plenum, not part of the chamber volume where gases are treated. Clean air is fed to this passageway 21 through input tube 23, which in this instance feeds uncontaminated air from a blower or compressor (not shown). Air from this passageway 21 is in turn fed down small tubes 27 generally in the center of, and on the surface of, vanes 18 and the flowing air feeds to and against source 11 which maintains the surface of source 11 clean of contaminants. At the central area of the reaction volume, lips 22 are formed on tubes 27 positioned on or in vanes 18 which direct the flowing gas (from the passageway 21) to create a controlled thin layer of uncontaminated air over the beam exit windows. This air prevents contaminants that might produce a corrosive compound from attacking the beam window or a compound that might cause deposits on the beam window as to reduce the effects of the electron beam flow through the window.

Source 11 releases electrons around its circumference and the gases that travel through pathway 17 are exposed at that stage to the flow of the most energetic of the output electrons. The gases that pass into the pie shaped sections 25 of the chamber and pass out through similar pie shaped segments 25 are also exposed in passing to the entire space distribution of the beam. In this way the gases travelling through the reaction volume are uniformly exposed to the electrons generated by source 11.

Flow control devices such as vanes, perforated planes, balance bars, or splitter plates may be employed to ensure a uniform distribution of gas flow within the reaction volume. Vanes are illustrated in this Figure.

The diameter of the reaction chamber 15, or more accurately the length of vanes 18 that define pie shaped segments 25, is controlled and determined by the energy of the electrons emitted from electron source 11. That energy determines the electrons' range. In the case of the use of a cylindrical tube as source 11, the high voltage applied to the electron beam tube and window thickness determine the energy of the electrons emitted.

The output of the tube is controlled by controlling the current emitted from the cathode and can be raised or lowered within its design limitations. This increases or decreases the dose delivered to the flowing gas. Dose applied depends on the power in the beam injected into the flowing gas and the flow rate of the gas through the reaction chamber.

The reaction chamber is designed to handle a maximum flow rate with the minimum of flow resistance through the system. In this embodiment, the upper limit of flow rate was designed to be 1200 cfm (2,000 m3/hour). The chamber in this case has a diameter of approximately 3 to 4 feet and an internal volume where gas is treated of approximately 13,000 cubic inches and an overall internal volume including input and output conduits and other connecting plenums in the system of approximately 28 cubic feet. System 15 also has a total weight with shielding (discussed below), of approximately 3000 pounds. Higher flow rates are possible but the power of a blower to drive the air stream through the system would have to increase non-linearly as one increases the flow rate. Alternatively, the entire system can be increased in size and capacity using the instant design as a base or in the case of a lower usage rate as for example where the chamber is used on the output vent of instrumentation using toxic materials, the design may be used to create a smaller system.

In the event that the contaminated gases require more dose than is available from the electron beam tube or source 11 operating at maximum output at the required flow rate of the air stream, additional systems may be included in the treatment facility and the contaminated gases would then be shared between systems. Alternatively, source can be constructed to generate a greater output. However, since one is dealing with in one instance vacuum tubes feeding electrons through a window, there is value to avoid attempting too high an output from such a source since one will encounter problems with the windows, cathodes, power supplies, etc. as one increases size and output requirements. In general a source with an output of several kilowatts of beam power is illustrated in the configurations discussed in these applications.

In FIG. 2 there is illustrated the reaction chamber 15 in a cutaway view that shows a single output plenum 26 connected between input opening 16 and input conduit 12. This plenum extends through output conduit 13. Also shown is the connection between opening 20 and output conduit 13. Although only a single input plenum 26 is shown, it will be understood from the illustration in FIG. 1, that a plurality (4 in FIG. 1) of these connecting plenums exist between the reaction volume and the input conduit 12 as to provide a flow path to each of the input openings 16 shown in every other section of the reaction volume of FIG. 1. In a like sense although only a single opening is shown between the output conduit and the volume of the reaction chamber, four holes with connecting tubes are strategically placed throughout the volume matching openings 20 in FIG. 1 connecting the volume of the chamber volume to the output conduit. A vane 18 is also shown in this Figure.

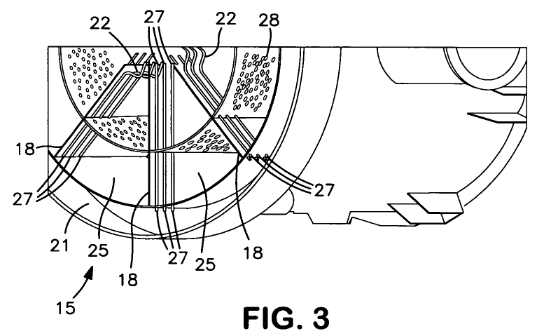

Referring now to FIG. 3, there is illustrated another cut away view that is intended to clarify aspects of chamber 15 shown and discussed in connection with FIGS. 1 and 2. Two pie shaped sections 25 can be seen in this Figure. Passageway 21 is seen extending around the outer rim area of the reaction chamber. Vanes 18 extend from the inner wall of outer passageway 21 to the central area near where the source or tube 11 is positioned. Along the surface of vanes 18 are a number of small tubes 27 that transport clean air used to prevent or reduce the deposit of material on the electron emitter windows or surface. In some applications of electron beam destruction, the incoming gas stream may also contain particulate matter that can become deposited on the windows of the electron beam emitting device. Such deposits, if built up sufficiently, would cause significant energy loss of the emerging electrons, thereby decreasing the electron energy and power available for treatment of the waste stream emissions. Further, if the contents are corrosive to the beam windows, the window material could become eroded, eventually causing pinholes leading to vacuum loss within the electron emitter device.

Tubes 27 comprise a series fine tubes as sources of clean air attached to vanes 18 of the pie sections 25 with output ends directed toward the surface of the emitter or source 11. The air flowing out of tubes 27 is flowed toward the surface of the source or in the case of vacuum tube with emitter windows to the surface of the emitter windows. Tubes 27 are located so that the air from the tubes, tends to create an isolating layer of air (an “Air Curtain”) over the window surface, and then flows out into the reaction volume or the pie shaped sections 25 from which the air is carried out of the reaction chamber through the exit openings 20 and then out of the exit conduit 13. Tips 22 can be seen at the ends of the tubes. These act to direct the gases flowing down the pie shaped sections 25 toward the adjacent pie shaped section 25 to return to the output openings 20 where the treated gases exit from the reaction volume and then exit the reaction chamber 15.

Air fed through the small tubes is driven by an external blower. The air is directed to flow across the surface of the tube window or windows to form a protective layer that prevents the contaminated air to be treated from flowing to the surface of the windows. The thickness of the protective layer is controlled by the rate of air flowing from the small tubes, and this can be adjusted by blower controls.

Air from the blower is also directed into passageway 21 through input tube 23. Air entering this passageway from the blower flows around the periphery of the entire reaction chamber and into the distal ends of small tubes 27 that open to this passageway. The cross sectional area of the passageway is designed to minimize the pressure drop throughout to assure that air flows uniformly through each of small tubes 27 along all the vanes 18.

The outside of the reaction chamber walls and the top and the bottom should be shielded with lead. The thickness of the shielding for a particular reaction chamber to bring leakage radiation to below natural background is determinable using a Monte Carlo program. The calculations should take into account the maximum high voltage that accelerates the electrons as well as the maximum beam current. Thus the amount of shielding will depend on the particular reaction chamber and its specifications. In the chamber described in this specification, the shielding thickness varied from ⅜″ to ½″. Thickness in each instance is dependent on energy and the magnitude of the beam intensity. Radiation leakage measurements were used to confirm that using the findings of the Monte Carlo program results in correct amounts of shielding.

If there is an application that contains corrosive compounds that could attack the internal walls of the reaction chamber, or if acids result from the breakdown of treated compounds (e.g.; compounds containing chlorine upon treatment will contain hydrochloric acid aerosols), either corrosive resistant metals can be used in the fabrication of the reaction chamber, or corrosive resistant coatings can be applied (e.g.; silicon carbide) to the surfaces of the chamber.

Internal dimensions of the reaction chamber 15 depend on the gas flow rate for the process and the energy of the electron beam. The number of pie shaped sectors, the size of the of the inner and outer plenums, and the geometry of the flow control devices are designed to minimize the pressure loss through the reaction chamber and to provide uniform flow of process gas past the electron beam source. For example, the gas flowing into and out of the pie shaped sectors and through the vertical tubes that connect to the input and output plenums flows at low velocity through a partition of metal perforated with a pattern of holes (28) designed to cause uniform and smooth gas flow toward and away from the electron emitter.

Other embodiments for introducing the gas to the reaction volume include a circumferential plenum and axial plenums located above and below the reaction volume.

Although exemplary embodiments of this invention have been shown and described, it will be understood by those skilled in the art that variations of the chamber structure and its operation may be employed depending on the particular application intended and that such structures will follow from the understanding imparted by the equipment illustrated and the discussion of its operation as to facilitate modifications that may be made in the mechanisms of the system and its operation without departing from the scope of the invention as defined in the appended claims.

What is claimed is:

1. A reaction chamber comprising
a circular housing enclosing a chamber volume,
an input conduit to feed fluids for treatment into the chamber volume,
an output conduit to feed treated fluids out of said chamber volume,
an extended cylindrical electron source positioned centrally and extending through the chamber volume,
a first set of passageways between said input conduit to feed incoming fluid from said input conduit into the reaction chamber volume,
a second set of passageways between said reaction chamber volume and said output conduit to feed fluids treated within the chamber volume to said output conduit,
fluid flow paths within said chamber volume to flow incoming fluids from said first set of passageways to said extended cylindrical electron source and then away from said source and to said second set of passageways exposing the fluid to be treated in the chamber volume to energetic electrons issuing from said cylindrical electron source, and
releasing said treated fluid out said output conduit.

2. The reaction chamber of claim 1 in which the walls, top and base are shielded with lead to protect against radiation exposure.

3. The reaction chamber of claim 1 in which the reaction chamber volume is made up of a plurality of pie shaped sections and in which alternate sections are connected to said first set of passageways and the flow is to said electron source.

4. The reaction chamber of claim 3 and in which pie shaped sections adjacent to said sections in which connected to said first set of passageways is connected to said second set of passageways.

5. The reaction chamber of claim 1 in which inner walls of said first set of passageways include a protective coating to protect said walls against caustic materials passing therethrough.

6. The reaction chamber of claim 3 in which there is interposed a perforated wall between the openings of said first set of passageways to said pie shaped sections and said electron source.

7. The reaction chamber of claim 6 in which the perforated wall also extends between said electron source and the opening in the reaction volume to said second set of passageways to said output conduit.

8. The reaction chamber in accordance with claim 1 including tubular connections to feed air to said source to form an air curtain across its surface.

9. The reaction chamber in accordance with claim 8 in which the air is fed to said source through tubes extending along vanes within the reaction chamber volume that define the pie shaped sections therein.

10. A reaction chamber to expose gases passing therethrough to energetic electrons, comprising
an enclosed cylindrical housing defining a reaction chamber volume,
partitions defining pie shaped sections within said chamber volume,
a centrally positioned extended cylindrical source to release electrons into said volume,
input pathways to feed gases for treatment into selected said pie shaped sections and to direct such gases to said centrally positioned cylindrical source where electrons collide with molecules and alter the compounds in the gas to form radicals that initiate reactions,
a second gas source to feed gases around the circumference of said cylindrical source,
output pathways to feed gases away from said cylindrical source in pie shaped sections adjacent to said pie shaped sections fed by said input paths, and
conduits connected to said output pathways as to feed treated gas out of the reaction chamber.

11. A reaction chamber in accordance with claim 10 in which a perforated wall is positioned in the path of the flow of the gases to and from said cylindrical source.

12. A reaction chamber in accordance with claim 11 in which said volume is shielded to make said reaction chamber radiation safe.

13. A method of treating a gas comprising flowing the gas to be treated into a reaction volume, directing the gas toward an extended cylindrical source of electrons in the center of the reaction volume issuing electrons circumferentially from said source, causing the gas to flow along a portion of the surface of said source while in near contact with said source, and flowing the gas away from said source and out of said reaction volume.

14. A method of treating a gas in accordance with claim 13 in which the said source creates energetic electrons and in which said gas is initially toxic and is treated to make said toxic gas environmentally more acceptable.

15. A method of treating a gas in accordance with claim 13 in which the said source creates energetic electrons and in which said gas initially has a noxious odor and is treated to improve the social acceptability of the smell of the gas.

16. A method of treating a gas in accordance with claim 13 including creating an air curtain around said extended cylindrical source protecting said source during treatment of treatable gases in said reaction volume.

17. A method of treating a gas in accordance with claim 13 in which the flowing gas flowing to and from said extended cylindrical source passes through a porous wall in its path smoothing the gas flow.
